(19) United States
(12) Patent Application Publication
(10) Pub. No.: US 2007/0106347 A1
(43) Pub. Date: May 10, 2007

(54) Portable medical and cosmetic photon emission adjustment device and method using the same

(76) Inventor: Wun-Chen Lin, Walnut, CA (US)

Correspondence Address: ROSENBERG, KLEIN & LEE, 3458 ELLICOTT CENTER DRIVE-SUITE 101, ELLICOTT CITY, MD, 21043, US

(21) Appl. No.: 11/269,530

(22) Filed: Nov. 9, 2005

(51) Int. Cl. A61N 5/06

(52) U.S. Cl. 607/88

(57) ABSTRACT

The present invention describes a portable medical and cosmetic photon emission adjustment device with the control of dose of photon and a method for using the same. A light source for medical treatment is positioned in a portable device to adjust the dose of photon emission by a dose adjustment device. The dose adjustment device has a microprocessor for processing and transmitting control signals and instruction signals. The dose adjustment device is used to control a plurality of light sources. The light source can be a high power light-emitting diode (LED) or a laser diode, and is combined with a set of lenses to achieve light convergence or light dispersion.

### BACKGROUND OF THE INVENTION

1. Field of the Invention

The present invention relates to a portable medical and cosmetic photon emission adjustment device for use in a certain medical light sources, and in particular to adjusting light emission time and light emission area to achieve the control of photon dose for medical and cosmetic purposes.

2. Description of Related Art

A conventional photomodulation device is widely used in medical treatment and applied to big-sized machines. Fields of application include, for example, cosmetology or treatment of retina disease.

As shown in FIG. 1, animal anomalous tissue is exposed to a matrix-typed light source 10 for medical treatment. The conventional device includes a power supply, a central processing unit (CPU) configured with a variety of light parameters, a protocol for medication, and a probe. The conventional device provides a plurality of light parameters of the light source 10 so that light beams emitted from the light source 10 having wavelength in a specific range are therapeutic. According to the conventional device, wavelength of light beams is in the range of 360 nm to 440 nm, 630 nm to 700 nm, 740 nm to 760 nm, or 800 nm to 1100 nm.

For therapeutic light beams, wavelength of light beams is in the range of 550 nm to 660 nm. The conventional device is shown in FIG. 2A and FIG. 2B. A light source 20 can emit light beams on all parts of a human body.

Reference is made to FIG. 3. FIG. 3 illustrates a method and device for stimulating hair growth. It provides a comb-liked emitter to split hair so that the emitter emits laser beams upon the epicranium. As shown in FIG. 3, the comb-like emitter includes a lower cover 1 and an upper cover 2. The emitter also includes two rows of teeth 3, a laser module 8, a switch unit 9, a coupling assembly 5 and a laser splitter/reflector 6. In this regard, laser beams are guided to the teeth 3 by the laser splitter/reflector 6 so that the laser beams emit from grooves of the teeth 3.

However, the laser beams are used as light source to stimulate hair growth by destroying skin tissue. Because of a thermal effect, wrong manipulation of the light source can cause skin to be burned. Besides, density of teeth of the emitter results in uneven laser beams on the affected part.

The conventional device is not widely used for cosmetology by individual because the conventional device employing therapeutic light beams is usually applied to big-sized medical instrument or appliance. Additionally, the conventional device does not control emission area so that it is impossible for the conventional device to control accurately the dose of photon emission. As the photon acts like medicine and the photon therapy gains more popular in cosmetic and medical applications, the control of dose of photon will become very important in photon therapy.

### SUMMARY OF THE INVENTION

It is an object of the present invention to provide a portable medical and cosmetic photon emission adjustment device with the control of dose of photon for use in a certain medical light source. A light source for medical treatment is positioned in a portable device so as to adjust dose of photon emission by a dose adjustment device. The dose adjustment device includes a microprocessor for processing and transmitting control signals and instruction signals. The dose adjustment device is electrically coupled to a configuration controller of the microprocessor to control a plurality of switch devices. Besides, the switch devices include a power switch, a time control switch, a light source control switch and an emission mode switch. The present invention utilizes a chip-on-board manufacturing process to manufacture a light source and includes an emission area adjustment device. The light source can be a high power light-emitting diode (LED) or laser diode and is combined with a set of lenses to achieve light convergence or light diffusion.

According to the present invention, the method for adjusting dose of photon emission includes the steps of determining how much a dose of photon emission is, adjusting an emission area adjustment device to change emission area, and adjusting emission time.

### BRIEF DESCRIPTION OF THE DRAWINGS

The present invention can be fully understood from the following detailed description and preferred embodiment with reference to the accompanying drawings, in which:

FIG. 1 schematically illustrates a light source of a conventional low power photon medical device;

FIGS. 2A and 2B illustrate one embodiment of the conventional medical device;

FIG. 3 schematically illustrates a conventional device for stimulating hair growth;

FIG. 4 schematically illustrates a portable medical and cosmetic photon emission adjustment device of the present invention;

FIG. 5 shows a block diagram of portable medical and cosmetic photon emission adjustment device according to the present invention;

FIG. 6 is a flowchart showing a method for adjusting photon emission of the present invention;

FIG. 7 illustrates one embodiment of a portable medical and cosmetic photon emission adjustment device of the present invention;

FIG. 8 illustrates a wearable-type photon emission adjustment device of the present invention; and

FIG. 9 illustrates a hat-type photon emission adjustment device of the present invention.

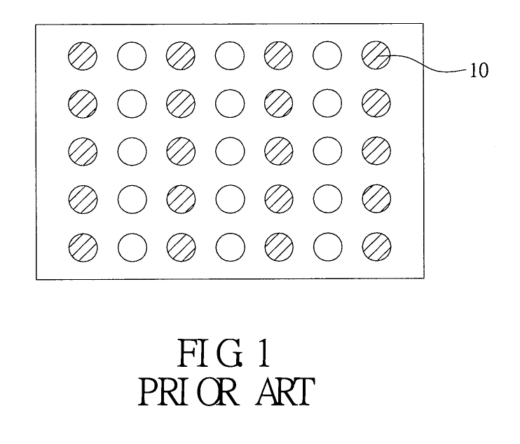

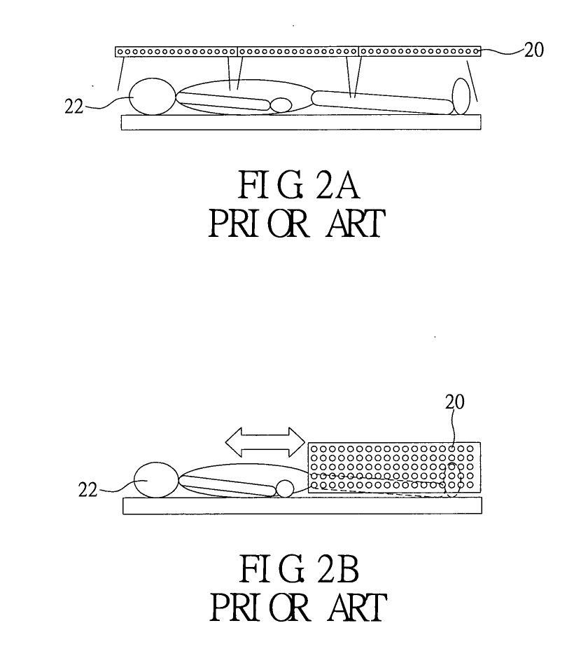

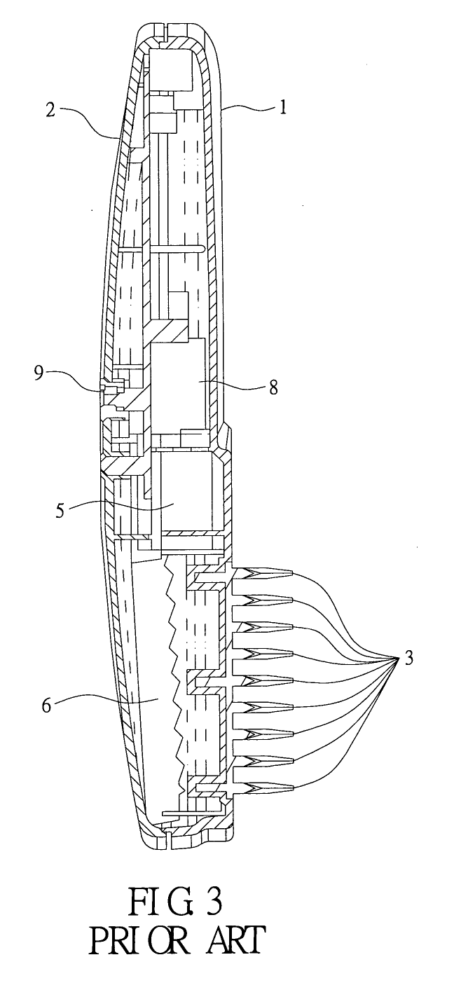

### DETAILED DESCRIPTION OF THE PREFERRED EMBODIMENTS

The following detailed description is of the best presently contemplated modes of carrying out the invention. This description is not to be taken in a limiting sense, and is made merely for the purpose of illustrating general principles of embodiments of the invention. The scope of the invention is best defined by the appended claims.

The present invention provides a portable medical and cosmetic photon emission adjustment device (hereafter referred to as “portable photon emission adjustment device”) and is a hand-held photon emission adjustment device. Besides, the present invention is used to determine a dose of photon emission. The present invention is also used to adjust an emission area adjustment device to change emission area and adjust emission time. The present invention utilizes adjustment of light source to adjust medical and cosmetic doses.

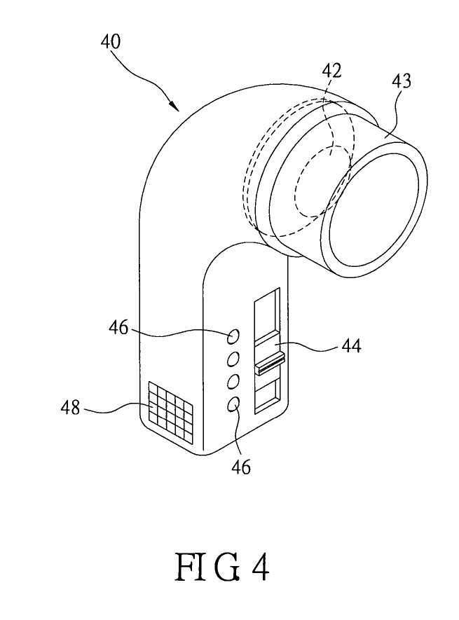

FIG. 4 schematically illustrates a portable photon emission adjustment device of the present invention. FIG. 4 shows a portable photon emission adjustment device 40 including a high power light source 42 and an emission area adjustment device 43. The high power light source 42 can be a laser diode, a super luminous light source or a light-emitting diode (LED). According to the present invention, the light beams emitted by the high power light source 42 having wavelength in a range are therapeutic, and the high power light source 42 is installed within the portable photon emission adjustment device 40. The light beams are controlled by the high power light source 42 of the emission area adjustment device 43. If the emission area adjustment device 43 is also positioned within the portable photon emission adjustment device 40, then the emission area is constant. If the emission area adjustment device 43 is external device, then the emission area varies due to the beam divergence of light source. The emission area will be depended on the total length between the emission area adjustment device 43 and the location of light source. It is convenient for user to grip one end of the emission area adjustment device 43.

Furthermore, the high power light source 42 of the present invention is manufactured by a chip-on-board (COB) manufacturing process, and the high power light source 42 can be a high power light-emitting diode or laser diode. A single light-emitting diode is packaged by the chip-on-board manufacturing process, and a plurality of light-emitting diodes is packaged by an array on board manufacturing process. In this regard, light emission is improved and the light beams with different wavelengths are emitted by the device to achieve medical and cosmetic purposes. As described above, the chip-on-board (COB) manufacturing process is an integrated circuit package process. The packaging process is used to place a chip directly on a printed circuit board or a substrate. The steps of the packaging process include (1) placing a chip, (2) connecting electrical wire and (3) applying packaging manufacturing. Thus, the volume of the device is significantly decreased and the output power intensity of photon is much higher than that of the conventional photomodulation device.

A configuration controller 44 is positioned in a grip portion of the portable photon emission adjustment device 40 so as to control the power switch, light emission time, light source control, light emission area and emission mode. A plurality of signaling lights 46 is positioned near the configuration controller 44 to inform a user of operation of the portable photon emission adjustment device 40. The portable photon emission adjustment device 40 also has a speaker 48 to remind the user of the operation of the portable photon emission adjustment device 40. These sounds include, for example, a warning sound for mis-manipulation, a sound for power-on or power-off, and a sound for device malfunction.

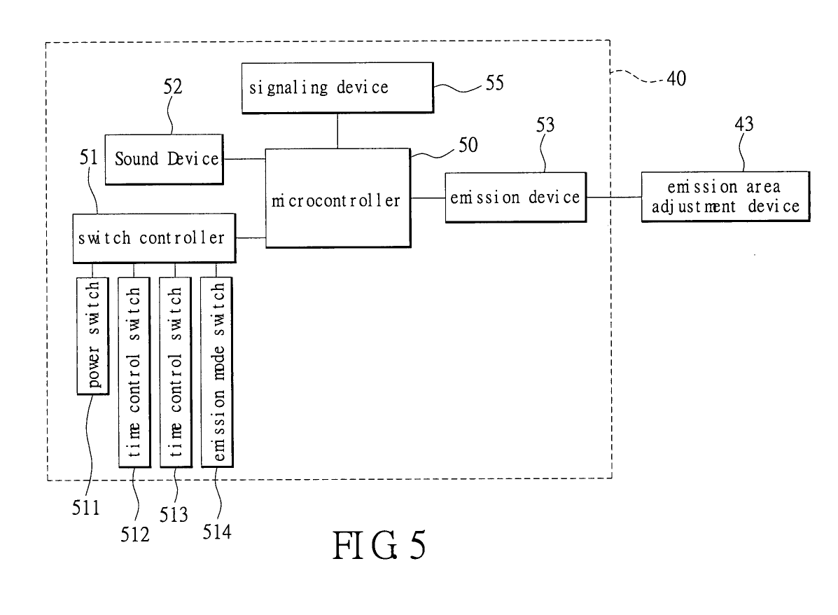

FIG. 5 illustrates a block diagram of portable photon emission adjustment device according to the present invention. The present invention includes a plurality of components electrically connected to each other. In addition to a power supply, the present invention includes a microcontroller 50, a configuration controller 51, and an emission device 53.

Besides, the configuration controller 51 is used to control a plurality of switches, including, as shown in FIG. 5, a power switch 511, a time control switch 513, and an emission mode switch 514. The portable photon emission adjustment device also has a signaling device 55 and a sound device 52 for indicating operations.

Reference is made to FIG. 5. The microcontroller 50 is used to control and process signals and communicates between signaling lights. Users can turn on or turn off the power switch 511 by the configuration controller 51, and the user can set up the emission time of the emission device 53 by the time control switch 512. Besides, the user can turn on or turn off the emission device 53 by a light beam control device 513. The present invention also provides combinations of different light beams, wavelengths of light beams and kinds of light beams, so as to adjust the dose of light beams. The present invention is used to configure the operation mode of the emission device 53, such as, for example, a continuous emission mode, a pulse emission mode, a cyclic emission mode or a bright/dark mode. The emission area adjustment device 43 is positioned where the portable photon emission adjustment device 40 connects to the emission device 53. Thus, the present invention provides different emission area and associated emission time to control the dose of light beams. In this regard, the present invention provides the correct dose of light beams in terms of accurate emission area and accurate emission time. Thus, an overdose or underdose of light beams will not happen. Additionally, the configuration controller 51 can be a plurality of electronically control buttons or menu switches.

As shown in FIG. 5, the microcontroller 50 receives instructions from the configuration controller 51 and sends a control signal to the emission device 53 to control the emission device 53. This includes, for example, functions of power supply, emission time, light beams control, and emission mode.

According to the present invention, the emission device 53 can be a laser diode, a superluminous light source, or a light-emitting diode, and the emission device 53 can be a combination of any kind of light source. The emission device 53 can be positioned in array or stand alone, and emits light beams having a combination of any wavelength. Besides, the emission device 53 may co-operate with a light diffuser to spread light beams on an anomalous tissue.

As described above, the microcontroller 50 is used to transmit the control signal to the signaling device 55 and the sound device 52 to remind users of the operation. The signaling device 55 can be comprised of a plurality of signaling lights to indicate the operation status, including, for example, blinking, change of brightness, or illumination in turn. The sound device 52 emits sounds, such as, for example, warning beeps for mis-manipulation, turn-on/turn-off, or device malfunction, to inform users.

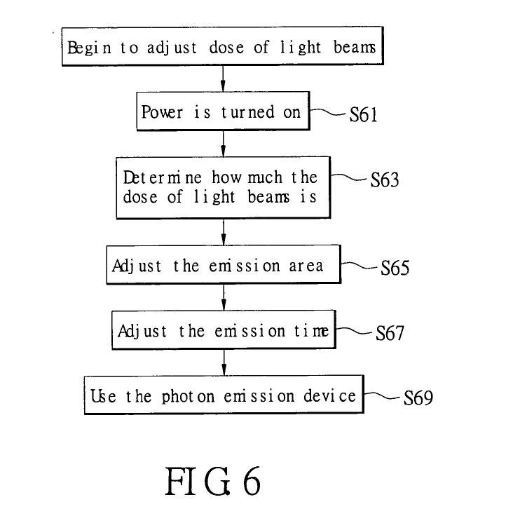

Reference is made to FIG. 6. FIG. 6 is a flowchart showing a method for adjusting photon emission of the present invention. The flowchart is described in detail below:

Step S61: In step S61, users utilize the portable photon emission adjustment device of the present invention to adjust emission of light beams. Users turn on the portable photon emission adjustment device.

Step S63: In step S63, the dose of light beams, an emission area and photon energy are determined.

Step S65: In step S65, the emission area can be adjusted by replacing or adjusting the emission area adjustment device of the present invention. The emission area adjustment device of the present invention includes a built-in device and one or several the external devices. The built-in device can adapt or connect to the external device that has different emission areas. Thus the emission area can be changed from a built-in device to an external device by adding the external device and vice versa

Step S67: In step S67, the time control switch and the emission mode are adjusted to get the dose of photon emission needed.

Step S69: In step S69, the present invention emits light beams on the users' skin at the area of interest.

According to the present invention, the dose of the photon emission is defined in terms of photon emission power, emission area and emission time. The equation of dose of the photon emission is:

D=(P*T)/A

where D is the dose of photon emission, P is the photon emission power, T is the emission time and A is the emission area. Energy of the photon is the product of the photon emission power and the emission time, so that the dose of the photon emission is defined as energy of the photon per unit area.

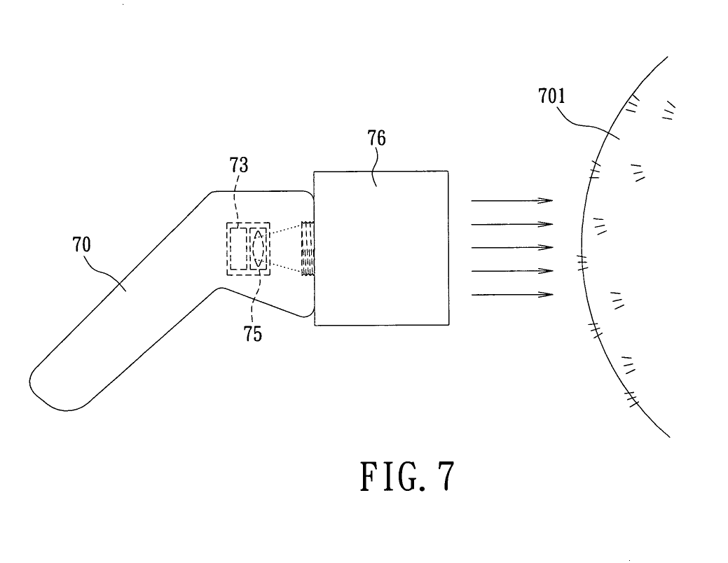

Reference is made to FIG. 7. FIG. 7 illustrates one embodiment of a portable photon emission adjustment device of the present invention. The device of the present invention is positioned in a portable device 70, and a light-emitting diode (LED) emitting therapeutic photon beams with wavelength in a certain range is used as a light source 73. A set of lenses 75 is positioned above the light source 73 so that light beams can be focused on a smaller area. Additionally, a diffuser function is integrated with the set of lenses 75, so that light beams can be evenly distributed over the skin and do not harm the skin. For example, the device of the present invention is used to stimulate hair growth. The device of the present invention provides the choice of continuous or pulse emission mode, adjusts the emission area to cover the user's skin area of interest, and adjusts the emission time to compensate the intensity changed and to adjust dosage and stimulate hair growth.

FIG. 7 shows a portable photon adjustment device 70 having at least one light-emitting diode/laser diode 73 as light source. The light-emitting diode/laser diode is packaged by a chip-on-board (COB) manufacturing process or by an array on board manufacturing process in another embodiment of the present invention. In addition to improve energy of the light beams, the present invention can emit light beams with a plurality of different wavelengths to meet cosmetic or therapeutic needs. The light-emitting diode/laser diode 73 emits therapeutic photon or light beams with wavelength in a range and is positioned on one side of the portable device 70 so that it is convenient for users to grip and operate. Additionally, the light-emitting diode/laser diode 73 can evenly distribute light beams by a set of lenses 75. A photon emission area adjustment device 76 is an external device with different emission area and is cylindrical and positioned along the direction of the light beams from the light-emitting diode/laser diode 73. The emission area is determined by the beam divergence of the light and the length of the photon emission area adjustment device 76. The longer the length of the photon emission area adjustment device 76 is, the larger area of the emission area is. If the photon emission area adjustment device 76 is a built-in device, then the emission area is fixed. For example, the light-emitting diode/laser diode 73 emits light beams onto hair 701.

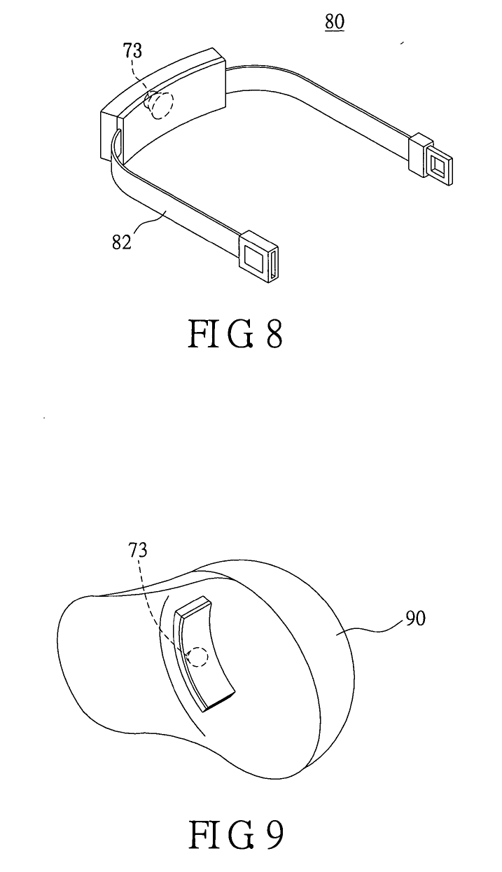

Reference is made to FIG. 8. FIG. 8 illustrates a wearable-type photon emission adjustment device of the present invention. As shown in FIG. 8, the light-emitting diode/laser diode 73 is positioned in a wearable-type device 80. Users wear the wearable-type device 80 like a hair hoop, by a flexible portion 82. The wearable-type device 80 is moved along the flexible portion 82 if necessary. Additionally, a light diffuser is positioned near the light-emitting diode/laser diode 73 so as to evenly distribute the light beams and increase emission area. Thus, skin burns can be avoided.

Reference is made to FIG. 9. FIG. 9 illustrates a hat-type photon emission adjustment device of the present invention. As shown in FIG. 9, the light-emitting diode/laser diode 73 is positioned within a hat-type device 90. It is convenient for users to receive medical treatment. A light diffuser is positioned near the light-emitting diode/laser diode 73 (indicated by a dashed line) so as to evenly distribute the light beams and increase emission area. Thus, skin burns can be avoided.

According to the present invention, dose control of the photon emission is necessary for the performance of cosmetic and medical treatment. If the dose of the photon emission is not sufficient, the treatment may not have expected result. If the dose of the photon emission is too much or overdose, it may have an adverse impact on skin. The dose of the photon emission is defined in terms of power of photon, emission area and emission time. Thus, it is necessary for the portable photon adjustment device to be integrated with capability of adjustment.

As described above, the light-emitting diode/laser diode is controlled by control circuit. Controllable entities include, for example, brightness of emission, wavelength of light beams, continuous emission, cyclic emission, and bright-to-dark/dark-to-bright mode.

According to the present invention, the portable device emits therapeutic light beams with a certain wavelength. Adjustable emission mode, emission time and emission area contribute to dose adjustment. The effect of the present invention includes, but is not limited to, cosmetics, growth of tissue, or medical treatment of injured eyes. Techniques and associated drawings of NASA Light-Emitting Diodes for the Prevention of Oral Mucositis in Pediatric Bone Marrow Transplant Patients, Pp. 319-324, Volume 20, Journal of Clinical Laser Medicine & Surgery are incorporated into reference.

While the invention has been described with reference to the preferred embodiments, the description is not intended to be construed in a limiting sense. It is therefore contemplated that the appended claims will cover any such modifications or embodiments as may fall within the scope of the invention defined by the following claims and their equivalents.

What is claimed is:

1. A portable medical and cosmetic photon emission adjustment device with the control of dose of photon, a dose of photon emission being defined in terms of power of photon, an emission area and an emission time, comprising:
a microprocessor, used to process and transmit control signals and instruction signals;
a configuration controller, being electrically coupled to the microprocessor to control a plurality of switch devices, including a power switch, a time control switch, a light source control switch and an emission mode switch;
a light source device, being electrically coupled to the microcontroller and used to emit therapeutic photon or light beams with a certain wavelength; and
an emission area adjustment device, being coupled to the light source device to control the emission area.

2. The photon emission adjustment device as claimed in claim 1, wherein the light source is a high power light-emitting diode (LED) or laser diode and is combined with a set of lenses to achieve light convergence or light diffusion.

3. The photon emission adjustment device as claimed in claim 1, wherein the light source is a laser diode.

4. The photon emission adjustment device as claimed in claim 1, wherein a set of lenses is positioned on the light source device.

5. The photon emission adjustment device as claimed in claim 4, wherein the set of lenses is a light diffuser.

6. The photon emission adjustment device as claimed in claim 1, wherein the light source device is manufactured by a chip-on-board (COB) manufacturing process.

7. The photon emission adjustment device as claimed in claim 1, wherein a plurality of light source devices is packaged by an array on board manufacturing process.

8. The photon emission adjustment device as claimed in claim 1, wherein the photon emission area adjustment device includes a built-in device and an external device.

9. A portable medical and cosmetic photon emission adjustment device, a dose of photon emission being defined in terms of power of photon, the emission area and emission time, comprising:
a microprocessor, used to process and transmit control signals and instruction signals;
a configuration controller, being electrically coupled to the microprocessor to control a plurality of switch devices, including a power switch, a time control switch, a light source control switch, and an emission mode switch;
a light source device, electrically coupled to the microcontroller, wherein the light source device is manufactured by a chip-on-board (COB) manufacturing process or by an array on board manufacturing process and is used to emit therapeutic photon or light beams with a certain wavelength; and
an emission area adjustment device, coupled to the light source device to control the emission area.

10. The photon emission adjustment device as claimed in claim 9, wherein a set of lenses is positioned at the light source device.

11. The photon emission adjustment device as claimed in claim 10, wherein the set of lenses is a light diffuser.

12. The photon emission adjustment device as claimed in claim 9, wherein a plurality of light source devices is packaged by an array on board manufacturing process.

13. The photon emission adjustment device as claimed in claim 9, wherein the photon emission area adjustment device includes and built-in device and an external device.

14. A method for adjusting photon emission, and dose of photon emission defined in terms of the emission area and emission time, the steps comprising:
determining how much the dose of light beams is;
adjusting the emission area, by replacing or adjusting the emission area adjustment device;
adjusting the emission time; and
emitting light beams on the users' skin of interest, wherein the light beams are therapeutic.

15. The method as claimed in claim 14, wherein operation mode includes a continuous emission mode, a pulse emission mode, a cyclic emission mode or a bright/dark mode.

16. The method as claimed in claim 14, wherein the light source is a high power light-emitting diode.

17. The method as claimed in claim 14, wherein the light source is a laser diode.

18. The method as claimed in claim 14, wherein the light source device is manufactured by a chip-on-board (COB) manufacturing process.

19. The method as claimed in claim 14, wherein a plurality of light source devices is packaged by an array on board manufacturing process.

20. The method as claimed in claim 14, wherein the photon emission area adjustment device includes a built-in device and an external device.
